(19) United States
(12) Patent Application Publication
(10) Pub. No.: US 2011/0301479 A1
(43) Pub. Date: Dec. 8, 2011

(54) System and Method for Assessing a Likelihood of a Patient to Experience a Future Cardiac Arrhythmia Using Dynamic Changes in a Biological Parameter

(75) Inventors: Raja N. Ghanem, Edina, MN (US); Paul J. DeGroot, Shoreview, MN (US)

(73) Assignee: Medtronic, Inc.

(21) Appl. No.: 13/152,508

(22) Filed: Jun. 3, 2011

Related U.S. Application Data
(60) Provisional application No. 61/351,169, filed on Jun. 3, 2010.
(60) Provisional application No. 61/351,222, filed on Jun. 3, 2010.

(51) Int. Cl. A61B 5/0452

(52) U.S. Cl. 600/515

(57) ABSTRACT

System and method for assessing a likelihood of a patient to experience a cardiac arrhythmia using dynamic changes in a biological parameter. A biological sensor is configured to sense a biological parameter of the patient. A processor is coupled to the biological sensor and is configured to determine a dynamic change of the biological parameter and determine the likelihood of the patient experiencing a cardiac arrhythmia based, at least in part, on the dynamic change of the biological parameter.

### RELATED APPLICATIONS

This application claims priority from U.S. Provisional Application No. 61/351,169, filed on Jun. 3, 2010, entitled “SYSTEM AND METHOD FOR ASSESSING A LIKELIHOOD OF A PATIENT TO EXPERIENCE A FUTURE CARDIAC ARRHYTHMIA USING DYNAMIC CHANGES IN A BIOLOGICAL PARAMETER”; and from U.S. Provisional Application No. 61/351,222, filed Jun. 3, 2010, entitled RISK STRATIFICATION SYSTEM DESCRIPTION.

### FIELD

The present invention is related to apparatus and methods for the assessment of risk of a cardiac arrhythmia and, especially to apparatus and methods for the assessment of risk of a cardiac arrhythmia by monitoring and/or measuring a biological parameter.

### BACKGROUND

Cardiac pacemakers, cardioverters and defibrillators are well known in the art and provide important life-saving treatment and safeguards for many patients. Such implantable medical devices have long been utilized to treat patients prone to suffering ventricular or atrial arrhythmias such as ventricular tachycardia and ventricular fibrillation. Once implanted in the patient's body, the cardiac pacemaker, cardioverter or defibrillator monitors the patient's heart. If the heart enters fast ventricular tachycardia or ventricular fibrillation, the cardioverter/defibrillator may deliver cardioversion therapy to shock the heart out of the tachycardia or fibrillation and return the heart to normal sinus rhythm.

Determining which patients may be effectively served by the implantation of an implantable cardioverter/defibrillator may be difficult. Historically, only patients who had previously suffered ventricular fibrillation were implanted with a cardioverter/defibrillator. Subsequent clinical testing and clinical trials have provided expanded indications for patients who may benefit from a cardioverter/defibrillator. However, these indications have typically been limited to patients who had suffered a previous medical condition, such as a myocardial infarction or heart failure. As such, a substantial portion of the population which has never suffered a ventricular fibrillation episode or other traumatic cardiac event has relatively few means for being indicated for an implantable cardioverter/defibrillator.

It is known, though, that patients who have never suffered a prior cardiac episode may still experience a ventricular or atrial arrhythmia such as ventricular tachycardia or ventricular fibrillation. Research has been directed toward analyzing cardiac signals to identify characteristics indicative of an increased propensity toward suffering cardiac arrhythmia such as ventricular or atrial tachycardia, or ventricular or atrial fibrillation and sudden cardiac death. Such characteristics include, for instance, the electrophysiological properties of cardiac tissue or triggers that may tend to lead to ventricular tachycardia or ventricular fibrillation. However, the results of such research has proven only partially successful, as the results of the studies have tended to show that a particular cardiac characteristic sampled at a particular time will tend to show only one aspect of the underlying cause of a future cardiac arrhythmia such as ventricular tachyarrhythmia or ventricular fibrillation. Thus, the tests based on cardiac characteristics have tended to provide a substantially incomplete estimation of the patient's likelihood of suffering a cardiac arrhythmia such as ventricular tachycardia or ventricular fibrillation.

### SUMMARY

In order to fit or equip patients who could be helped by a cardiac pacemaker, cardioverter and/or defibrillator, it would be desirable to have a more accurate indicator of which patient or patients are most at risk of cardiac arrhythmia such as fast ventricular tachycardia and/or ventricular fibrillation.

It has been determined that past practices, which have tended to measure physiological parameters and compare the measured physiological parameters against predetermined thresholds, may provide relatively weak predictive information. Rather, it has been determined that measurements which are sensitive to the dynamic change in physiological parameters may be relatively more indicative of a likelihood of a patient experiencing an arrhythmia in the future. In particular, a trend in a physiological parameter may, at times, be more predictive of future arrhythmias than the instantaneously measured value of the physiological parameter.

In certain circumstances, physiological parameters such as T-wave alternans, measured according to various methods, heart rate turbulence, T-wave alternans corresponding to a baroreflex response and deceleration capacity of a heart of the patient may be physiological parameters which may serve as effective markers for future arrhythmia. While such parameters may be measured instantaneously and compared against predetermined thresholds, dynamic changes in the parameters may tend to be more predictive than a comparison of the instantaneous measurement against a threshold. For instance, a patient for whom an instantaneous T-wave alternans measurement indicates a risk of future arrhythmia owing to the instantaneous measurement being outside of an allowable range may be determined to be at a reduced risk of future arrhythmia because the dynamic change in the patient's T-wave alternans show changes suggesting that T-wave alternans are not indicative of an underlying condition that may predispose the patient to a relatively high risk of ventricular arrhythmias.

Devices for the collection of various kinds of cardiac data, such as Holier monitors for the collection of electrical data, are known in the art. Further, implantable sensors have been developed which allow for cardiac monitoring in a manner similar to that of a Holter monitor but without the ongoing inconvenience to the patient created by external devices. In addition, implantable cardiac therapy devices such as pacemakers, defibrillators, implantable loop recorders and the like have long been provided with the capacity to sense and store cardiac data for subsequent analysis as well as to transmit diagnostic data telephonically or in real time. Any or all such devices may be utilized to sense physiological parameters of the patient and analyze the dynamic changes in the physiological parameters.

In an embodiment, a system for assessing a likelihood of a patient to experience a future cardiac arrhythmia comprises a biological sensor configured to sense a biological parameter of the patient and a processor operatively coupled to the biological sensor. The processor is configured to determine a dynamic change of the biological parameter and determine the likelihood of the patient experiencing a cardiac arrhythmia based, at least in part, on the dynamic change of the biological parameter.

In an embodiment, the processor determines the dynamic change based, at least in part, on the dynamic change of the biological parameter from a first time to a second time.

In an embodiment, the processor determines the dynamic change of the biological parameter further based, at least in part, on a rate of change of the biological parameter.

In an embodiment, a system for assessing a likelihood of a patient to experience a cardiac arrhythmia comprises a biological sensor and a processor. The sensor is configured to sense a biological parameter of the patient at a first time, then sense the biological parameter of the patient at a second time, the second time being later than the first time. The processor is operatively coupled to the biological sensor and configured to determine a change of the biological parameter from the first time to the second time. The system determines the likelihood of the patient experiencing a cardiac arrhythmia based, at least in part, on the change of the biological parameter.

In an embodiment, the processor determines the change of the biological parameter further based, at least in part, on a rate of change of the biological parameter.

In an embodiment, the biological parameter is at least one of a T-wave alternan, a T-wave alternans associated with a baroreflex response, heart rate turbulence and deceleration capacity of a heart of the patient.

In an embodiment, the likelihood of the patient experiencing a cardiac arrhythmia is relatively lower when a value of the T-wave alternan decreases from the first time to the second time than when the value of the T-wave alternan does not decrease from the first time to the second time.

In an embodiment, the likelihood of the patient experiencing a cardiac arrhythmia is relatively lower when a value of the T-wave alternan associated with the baroreflex response increases from the first time to the second time than when the value of the T-wave alternan associated with the baroreflex does not increase from the first time to the second time.

In an embodiment, the likelihood of the patient experiencing a cardiac arrhythmia is relatively lower when a value of the heart rate turbulence slope increases from the first time to the second time than when the value of the heart rate turbulence does not increase from the first time to the second time.

In an embodiment, the likelihood of the patient experiencing a cardiac arrhythmia is relatively higher when a value of the deceleration capacity decreases from the first time to the second time than when the value of the deceleration capacity does not decrease from the first time to the second time.

In an embodiment, the likelihood of the patient experiencing a cardiac arrhythmia is relatively higher when a value of the ejection fraction does not increase from the first time to the second time than when the value of the ejection fraction decreases from the first time to the second time.

In an embodiment, the second time is approximately six hours after the first time.

In an embodiment, the system determines the change of the biological parameter based, at least in part, on a difference in a value of the biological parameter at the first time and the second time.

In an embodiment, the value of the biological parameter at the first time corresponds to a maximum value of the value of biological parameter measured over a first period of time and the value of the biological parameter at the second time corresponds to a maximum value of the biological parameter measured over a second period of time.

In an embodiment, each of the first period of time and the second period of time are approximately twenty-four hour periods.

In an embodiment, the value of the biological parameter at the first time corresponds to an average value of the biological parameter measured over a first period and the value of the biological parameter at the second time corresponds to an average value of the biological parameter measured over a second period of time.

In an embodiment, each of the first period of time and the second period of time are approximately two hour periods.

In an embodiment, a method for assessing a likelihood of a patient to experience a future cardiac arrhythmia with an implantable device system comprising an implantable sensor and a processor, comprising the steps of sensing a biological parameter of the patient with the sensor at a first time, then sensing the biological parameter of the patient with the sensor at a second time. Then a change of the biological parameter from the first time to the second time is determined with the implantable device system and the likelihood of the patient experiencing a cardiac arrhythmia is determined with the implantable device system based, at least in part, on the change of the biological parameter.

In an embodiment, the determining the change step is based, at least in part, on a change of the biological parameter from the first time to the second time.

In an embodiment, the determining the change step is further based, at least in part, on a duration between the first time and the second time.

In an embodiment, each of the sensing steps occur after the patient has suffered a myocardial infarction.

In an embodiment, each of the sensing steps occur after the patient has been hospitalized.

In an embodiment, the biological parameter is measured continuously.

### FIGURES

FIG. 1 is an image of a torso of a patient;

FIG. 2 is an image of an implantable device;

FIG. 3 is a block diagram of the implantable device of FIG. 2;

FIG. 4 is a flowchart of a method of utilizing categorized markers to assess patient risk;

FIG. 5 is exemplary of a cardiac complex of a patient;

FIG. 6 is an application of a heart rate turbulence to an electrogram signal;

FIGS. 7a-7c are graphical depictions of an analysis of phase rectified signal averaging;

FIG. 8 is a flowchart for conducting the phase rectified signal averaging analysis illustrated in FIGS. 7a-7c;

FIG. 9 is a flowchart for analyzing heart rate turbulence in a patient;

FIG. 10 is a graphical depiction of a T-wave alternans analysis using a modified moving average;

FIG. 11 is a flowchart for conducing the T-wave alternans modified moving average analysis illustrated in FIG. 10;

FIG. 12 is a flowchart for performing a qualitative assessment of patient risk;

FIG. 13 is a flowchart for performing a quantitative assessment of patient risk;

FIG. 14 is a graphical illustration of more continuous monitoring by the implantable device of FIG. 2; and

FIG. 15 is a flowchart for utilizing the dynamic change of markers to determine a likelihood of a future arrhythmia.

### DESCRIPTION

The entire content of provisional U.S. Provisional Application Ser. No. 61/351,169, filed Jun. 3, 2010, and U.S. Provisional Application Ser. No. 61/351,222, filed Jun. 3, 2010 are hereby incorporated by reference.

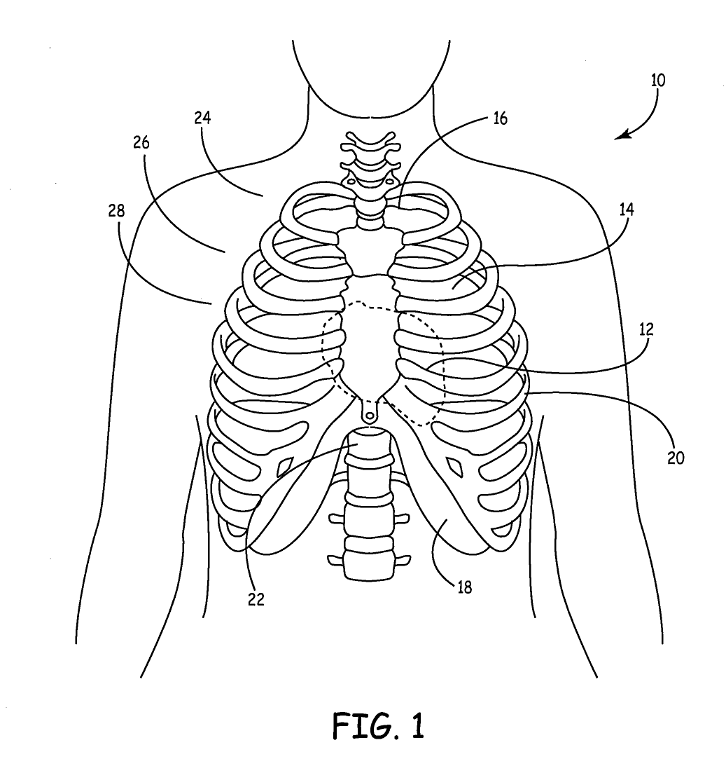

FIG. 1 is a cutaway drawing of patient 10. Heart 12 is positioned in thoracic cavity 14. Thoracic cavity 14 is commonly understood in the art to be bounded by thoracic inlet 16, diaphragm 18, ribs 20 and spine 22. Patient skin 24, musculature 26 and subcutaneous tissue 28 between skin 24 and musculature 26 are commonly not understood to be part of thoracic cavity 14.

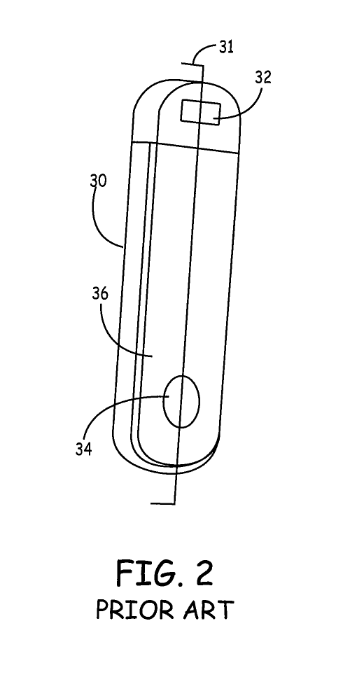

FIG. 2 is an image of implantable device 30. Implantable device 30 may be configured to stratify risk of heart 12 experiencing a cardiac event without meaningful risk of interruption in the collection of patient data and with greater permanence than may be provided with alternative devices, as disclosed, for instance, in U.S. Pat. No. 5,987,352, Klein et al, incorporated herein in its entirety. In various embodiments, implantable device 30 has a length along primary axis 31 from three (3) to six (6) centimeters and has a diameter less than or equal to one (1) inch (2.54 centimeters). In an embodiment, implantable device 30 has a length of approximately four (4) centimeters and a diameter orthogonal to primary axis 31 of one-half (0.5) inch (1.27 centimeters). In various embodiments, implantable device 30 is configured for subcutaneous implantation, which is known in the art to involve implantation of implantable device 30 under skin 24 but outside of thoracic cavity 14 of patient 12. In various embodiments, implantable device 30 may be implanted in tissue 28. Implantable device 30 can also be implanted sub-muscularly, that is below musculature 26, but outside of thoracic cavity 14.

Implantable device 30 may have electrodes 32, 34 at opposing ends of housing 36 along primary axis 31 of implantable device 30. In various alternative embodiments, electrodes 32, 34 are positioned on leads which extend from housing 36. In certain embodiments, the leads are similarly positioned subcutaneously. In alternative embodiments, the leads are transvenous and extend through vasculature of patient 10 and into heart 12. In various embodiments, electrodes 32, 34 are positioned a predetermined distance apart. In an embodiment, the spacing is equal to the length of implantable device 30. In alternative embodiments, electrodes 32, 34 are positioned at a distance of less than the length of implantable device 30. When implanted subcutaneously, electrodes 32, 34 may sense far-field electrical activity of heart 12 which may be interpreted in order to characterize the electrical and physical activity of heart 12.

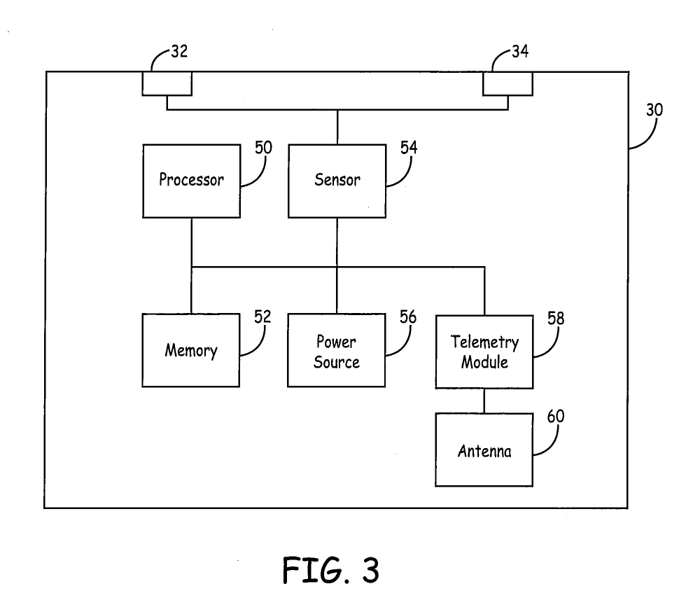

FIG. 3 is a block diagram of implantable device 30. Processor 50 provides computing and controlling functions for implantable device 30. Memory 52 stores data both stored through user input and sensed by implantable device 30 by way of electrodes 32, 34. Sensor 54 is coupled to electrodes 32, 34 and utilizes data sensed by electrodes 32, 34 to identify conditions of heart 12. In various embodiments, the function of sensor 54 is merely an aspect of the overall functionality of processor 50, and as such sensor 54 is not independent circuitry. In alternative embodiments, sensor 54 is separate componentry. Power source 56 provides power to the componentry of implantable device 30. In an embodiment, power source 56 is selected from conventional batteries well known in the implantable medical device art. In alternative embodiments, power source 56 is an alternative source of long-term power, such as a super capacitor. Telemetry module 58 is coupled to antenna 60 which, when placed in proximity of an external receiver, is configured to transmit data from processor 50, memory 52 or sensor 54 to an external device. In an embodiment, antenna 60 is an inductive coil configured to transmit data by way of an inductive field.

As cardiac signals are detected by electrodes 32, 34 and sensed by sensor 54, the data representing the cardiac signals may be stored in memory 52 and/or processed in processor 50. Alternatively, data representing the cardiac signals are transmitted the external device by way of telemetry module 58 without storage in memory 52 or processing in processor 50. In such embodiments, the external device performs the processing functions.

In order to stratify risk accurately, multiple “markers” or indicators of a cardiac condition or cardiac performance of patient 10 may be utilized together to obtain a relatively more complete evaluation of the condition of heart 12 than may be possible or practical to obtain on the basis of one measurement or marker. Taken together, multiple markers may help to obtain a risk stratification of a propensity of patient 12 toward suffering a future ventricular or atrial arrhythmia such as ventricular tachycardia or ventricular fibrillation. The risk stratification may rely not on one narrowly focused cardiac characteristic, but instead upon multiple characteristics that characterize different aspects of heart 12.

A measurement of an electrogram detected by electrodes 32, 34 positioned subcutaneously in patient 10 may generally be influenced by a relatively broad region of patient 10. Included in such broad region may be musculature 26 and the lungs of patient 10. Measurements detected with electrodes 32, 34 may be sensitive to signals generated by musculature 26 and lungs, as well as from heart 12, and are commonly referred to as far-field measurements.

In addition, measurements may be taken of non-electrical characteristics of patient 10, including, but not limited to, genetic analysis of patient 10, generally, and heart 12, specifically. Such analysis may include analysis of the patient's genes to identify mutations in heart 12, and may include analysis of the family history of patient 10 to identify increased risk of future cardiac disease.

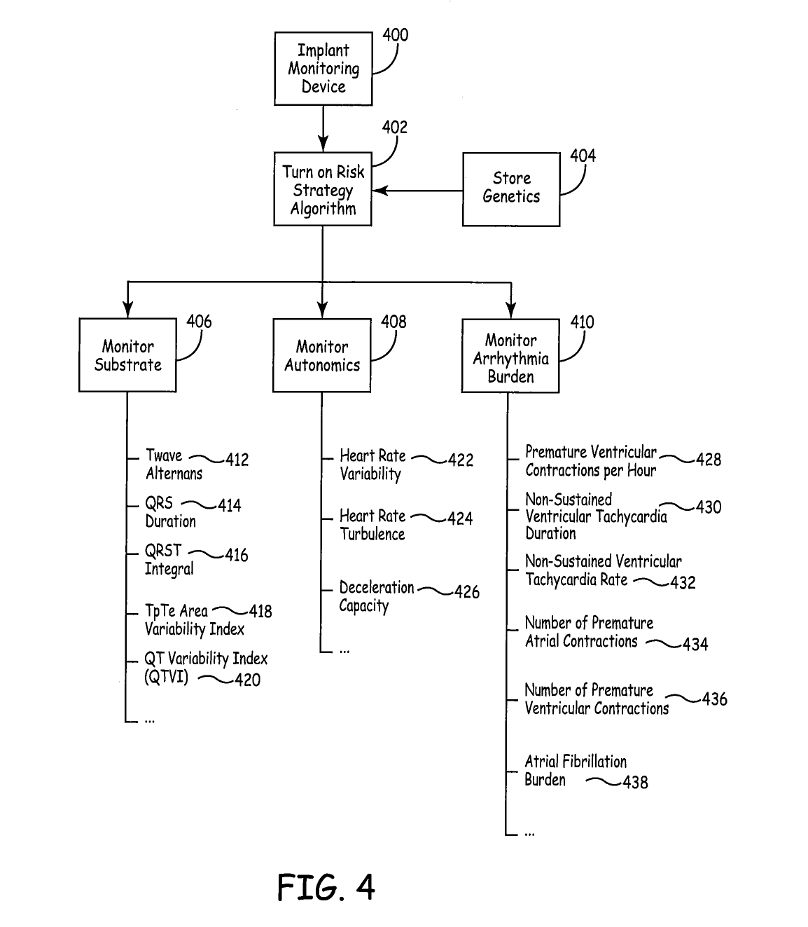

FIG. 4 is a flow chart illustrating a method of utilizing implantable device 30 to obtain data useful in stratifying risk of sudden cardiac death in a patient. Implantable device 30 is implanted (400) in patient 10. A risk stratification algorithm, shown below, may be turned on or otherwise enabled (402), in an embodiment in implantable monitoring device 30, in an alternative embodiment with a separate computing device such as a personal computer, proprietary programming or diagnostic device, and servers and processors located off-site, such as cloud computing systems and software-operated virtual machines. In one embodiment, genetic information may be obtained and provided to the risk stratification algorithm, in various embodiments by being stored (404) in memory module 52 or in a memory of a separate computing device.

In various embodiments, cardiac data is then collected which may be utilized by the risk stratification algorithm. In an alternative embodiment, the data may be collected without first turning on (402) the risk stratification algorithm. In such an embodiment, the data may be collected and then inputted into the risk stratification algorithm after the risk stratification algorithm is turned on. The cardiac data which may be collected includes data related to a cardiac substrate of heart 12, an autonomic system of heart 12, and, in the event the patient experiences an arrhythmia of some kind, data related to the burden of the arrhythmia on patient 10 generally, referred to as the “arrhythmia burden”.

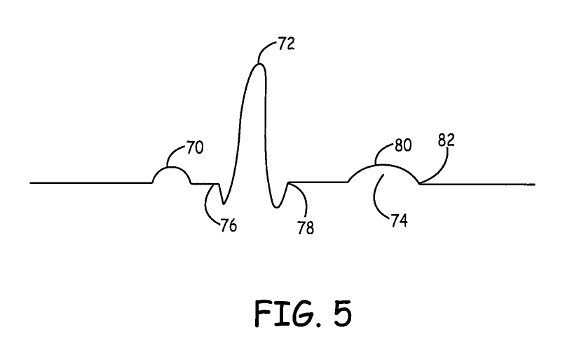

The substrate of heart 12 is monitored (406) for relevant data. A cardiac complex detected as part of an electrocardiogram is illustrated in FIG. 5. P-wave 70 represents a depolarization of the atria of heart 12. QRS complex 72 represents a repolarization of the atria of heart 12 and a depolarization of the ventricles of heart 12. T-wave 74 represents the repolarization of the ventricles of heart 12. In the embodiment of implantable device 30, electrodes 32, 34 are configured to detect the electrical signal representative of the cardiac complex and sensing module 54 is configured to interpret the electrical signals sensed by electrodes 32, 34.

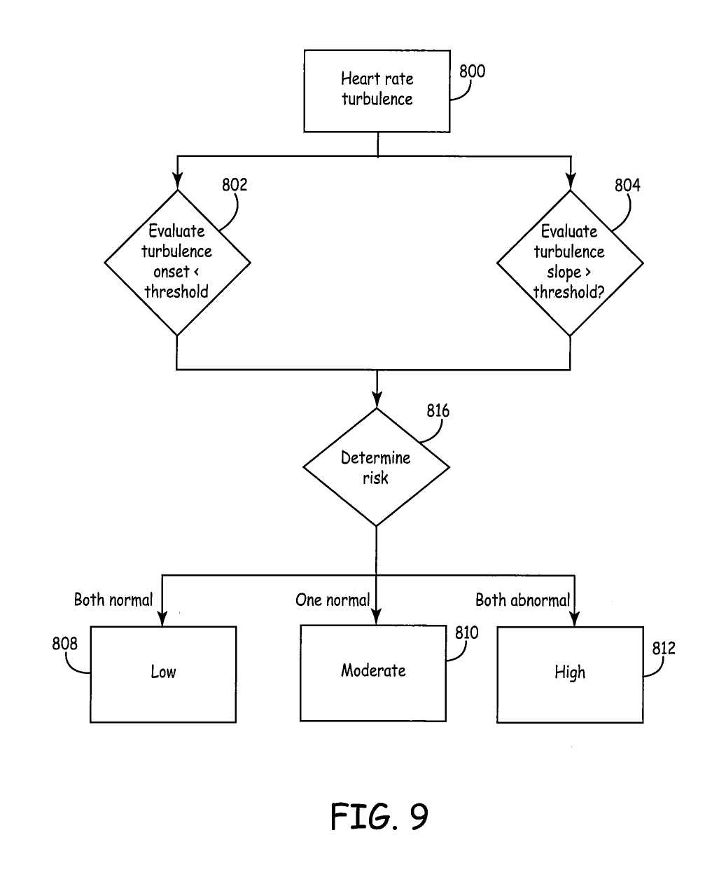

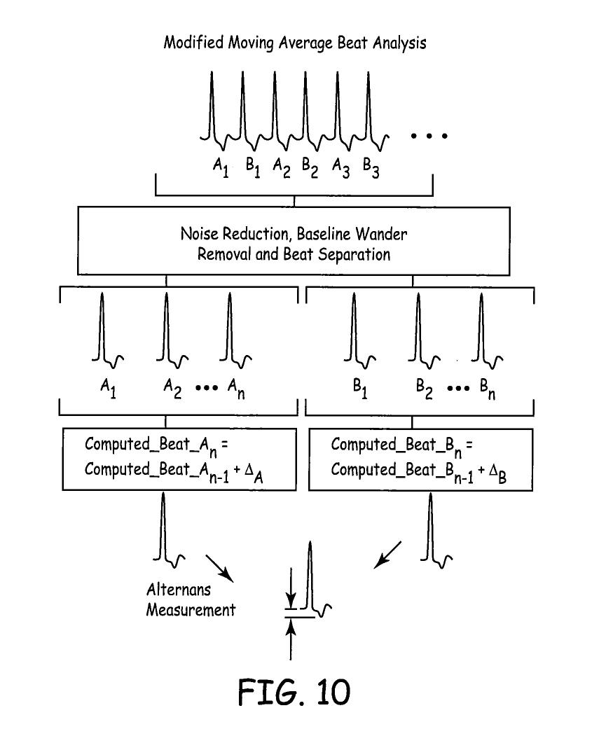

Examples of data related to the cardiac substrate include data related to T-wave 74 alternans (412), which accounts for beat-to-beat variability, often cyclic alternating variability, in T-waves 74 (FIGS. 9 and 10 below). Further substrate data monitored may include a duration (414) of QRS complex 72 from QRSₛₜₐᵣₜ 76 to QRS_{end} 78, and an integral (416) of a QRST complex, defined as the area under each of QRS complex 72 and T-wave 74.

In various embodiments, the QRST integral (416) may be compared against a threshold value which is fixed in percentage terms of occurrence but which is dynamic in precise value. In certain embodiments, the threshold is the largest twenty-fifth percentile of all QRST integrals. In an embodiment, the threshold is the largest twenty-fifth percentile of all QRST integrals measured in a learning dataset compared against a fixed threshold. Alternatively, the QRST integral (416) is compared against a fixed threshold generally. In further alternative embodiments, the QRST integral is assessed dynamically against changes in the QRST integral (416) over time. This analysis thereby evinces a dynamic change in the QRST integral (416).

Further, an area (418) of T-wave 74 may be computed by integrating the T-wave from Tₚₑₐₖ 80 to T_{end} 82. Such a measurement may be indicative of a likelihood that a patient will experience fast ventricular tachycardia and/or ventricular fibrillation. A use for T-wave area (418) is described in an abstract by Larisa G. Tereshchenko et al., entitled Tₚₑₐₖ-T_{end} Area Variability Index from Far-Field Implantable Cardioverter-Defibrillator Electrograms Predicts Sustained Ventricular Tachyarrhythmia¹, incorporated here by reference in its entirety. Increased variability of Tₚₑₐₖ-T_{end} area index may provide a measure of both alternating and non-alternating repolarization instability, and may predict sustained ventricular tachycardia or ventricular fibrillation events in patient 10. ¹ Tereshchenko et al., “Tpeak-Tend Area Variability Index from Far-Field Implantable Cardioverter-Defibrillator Electrograms Predicts Sustained Ventricular Tachyarrhythmia”, Heart Rhythm, vol 4, no. 5, May Supplement 2007.

Further, a variability (420) in time between QRSₛₜₐᵣₜ 76 to T_{end} 82 may be measured as a Q-T variability index. An example of a use for a Q-T variability index is described in U.S. Pat. No. 5,560,368, Berger, incorporated here by reference in its entirety. A template Q-T interval may be created based on QRSₛₜₐᵣₜ 76 to T_{end} for one cardiac cycle. An algorithm is then utilized to determine the QT interval of other cardiac cycles by determining how much each cycle must be stretched, i.e. elongated, or compressed in time so as to best match the template.

In an embodiment, all of the substrate data described above are utilized. In alternative embodiments, additional data related to the cardiac substrate may be incorporated. In alternative embodiments, fewer than all of the recited substrate data are utilized. In an embodiment, T-wave alternans (412) and the QRST integral (416) are utilized. In an embodiment, only T-wave alternans (412) are utilized.

Autonomics of heart 12 are likewise monitored (408). Examples of data related to autonomics, i.e., data related to the automatic nervous system, include heart rate variability (422), heart rate turbulence (424) and deceleration capacity (426). Heart rate variability (422) may be an index of variability in sequential normal heart beats. Heart beats may be identified on the basis of common points during the cardiac complex of each beat. In an embodiment, a time between consecutive beats is defined as the time between Rₚₑₐₖ 84 of consecutive complexes. Heart rate turbulence may reflect an immediate acceleration in heart rate followed by recovery after an occurrence of a premature ventricular contraction. Deceleration capacity may be defined as a baseline autonomic tone of patient 10 measured from the heart rate deceleration (that is, decreases in heart rate) over an extended period, typically twenty-four (24) hours. In certain embodiments, deceleration capacity may serve as a contemporary analog to heart rate variability.

In an embodiment, heart rate turbulence (424) refers to the cycle length fluctuations for a number of “normal” heart beats following a premature ventricular contraction or beat. In an embodiment, heart rate turbulence (424) is based on beats following a single premature ventricular contraction which meet certain stability criteria. In an embodiment, the interval 86 between normal beats must be greater than three hundred (300) milliseconds and less than two thousand (2000) milliseconds. In various embodiments, measurements for heart rate turbulence (424), as well as other markers such as T-wave alternans (412), do not occur during episodes of fast ventricular tachycardia, ventricular tachycardia, atrial tachycardia, atrial fibrillation and other unstable or arrhythmic events. In an embodiment, if an atrial tachycardia or atrial fibrillation episode is not in progress at the start of a two (2) minute period, cardiac events are processed for that two (2) minute period. If an atrial tachycardia or atrial fibrillation episode is detected at the end of the two (2) minute period, the data for that two (2) minute period may be discarded. Note that data for a two (2) minute period that ends with a detection of atrial tachycardia or atrial fibrillation episode termination may also not be used, because an atrial tachycardia or atrial fibrillation episode is in progress at the start of that two (2) minute period.

In various embodiments, the number of sinus beats utilized for computation of heart rate turbulence (424) range from five (5) beats to twenty (20) beats. In an embodiment, the number of beats is sixteen (16) beats. In sinus rhythm, the heart rate may accelerate after the premature beat and then recover to a baseline value over several beats. This adaptation of heart rate to a premature ventricular contraction (PVC) may be absent in high risk patients. Mechanistically, heart rate turbulence may be due to a transient loss of vagal efferent activity due to missed baroreflex afferent input following a premature beat. A drop in blood pressure following a premature beat is sensed by a baroreflex receptor of patient 10 which then inhibits a vagal tone of patient 10, resulting in early acceleration of a cardiac cycle length. The inhibition may die out over several beats thereafter and as the blood pressure recovers to normal levels, the baroreflex receptor is reloaded and vagal activity is restored.

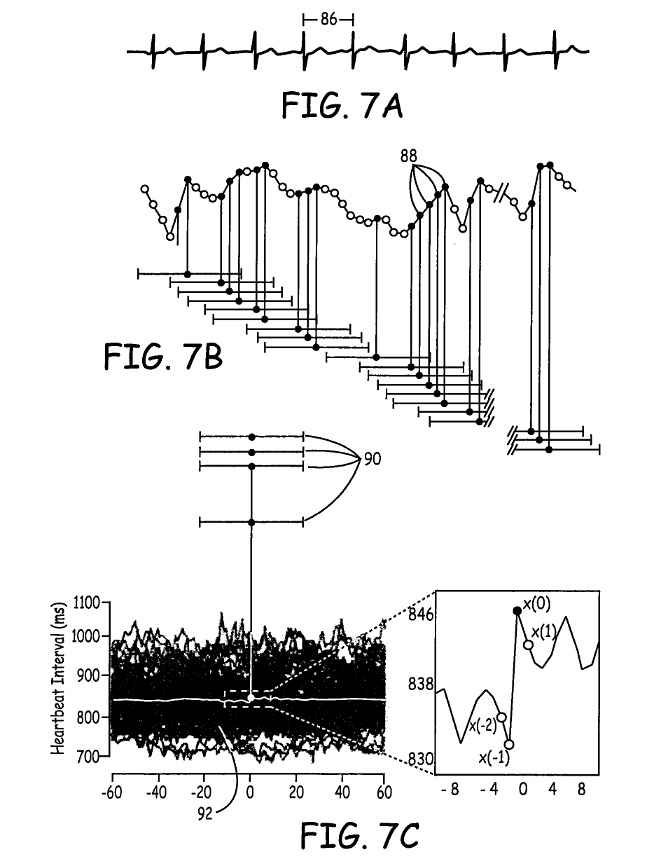

Heart rate turbulence is commonly derived from twenty-four hour electrocardiogram Holter recordings but may also be derived from a more continuous and longer-term monitor, such as implantable device 30 as described herein. Like heart rate variability, heart rate turbulence is computed from a plot of heart rate intervals 86 (FIG. 7a) and a heart beat number, known in the art as a tachogram. Heart rate turbulence may be characterized by two variables: turbulence onset and turbulence slope. In an embodiment, turbulence onset is defined as the difference between the mean of the first two intervals 86 of consecutive complexes after the premature ventricular contraction and the mean of the last two sinus intervals 86 of consecutive complexes preceding the premature ventricular contraction divided by the mean of the last two sinus intervals 86 of consecutive complexes preceding the premature ventricular contraction.

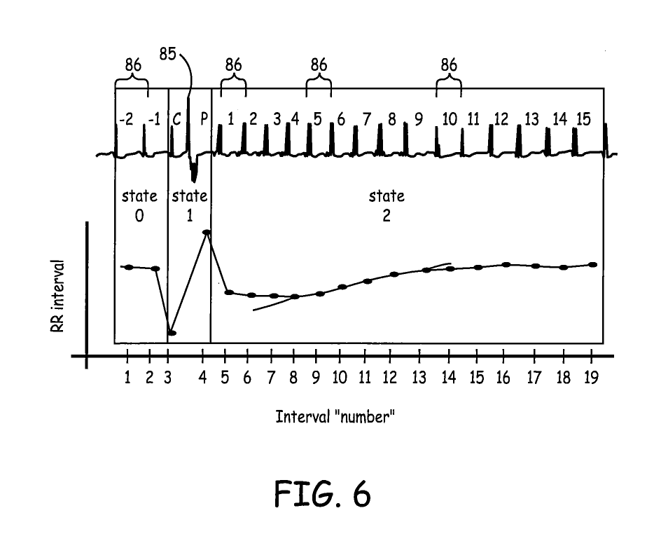

Such heart rate turbulence is illustrated in FIG. 6, in which an electrogram is divided into state 0, state 1 and state 2. State 0 represents the phase before premature ventricular contraction 85. In state 0, the algorithm may attempt to collect two intervals 86 followed by premature ventricular contraction 85, i.e., a beat categorized as premature. When premature ventricular contraction 85 occurs which is preceded by intervals 86 fulfilling the stability criteria described above, state 1 is entered. When premature ventricular contraction 85 is followed by a compensatory pause, i.e., a beat categorized as late, (interval 86 marked as P), the state may be set to state 2. If interval P is not a compensatory pause, then the state may be set to state 0 and the collection of intervals 86 resumes. In state 2, a number of intervals 86 may be collected as described above. When one of the sixteen (16) beats, in an embodiment, is categorized as early, late or not usable, then the state may be changed to state 0 and the collection intervals 86 resumed. If a beat interrupting the state 2 interval 86 sequence is a premature ventricular contraction 85 and there are two (2) or more natural intervals 86 between the previous premature ventricular contraction 85 and the current premature ventricular contraction 85, then the two (2) events prior to the current PVC and the current premature ventricular contraction 85 may be re-evaluated for temporal behavior based on the R-R median at the current premature ventricular contraction 85 as described above. If the intervals 86 meet the criteria, the state may be changed to state 1 and the two (2) intervals 86 before the interrupting premature ventricular contraction 85 are used as the two (2) intervals before the premature ventricular contraction 85.

In an embodiment, upon completion of a heart rate turbulence (424) test, monitoring returns to normal until the next detected premature ventricular contraction 85, when the test is repeated. When data collection for the heart rate turbulence (424) is complete the segment may be added to the previous segments, i.e., a summation tachogram containing the nineteen (19) summation values for all intervals 86.

In alternative embodiments, turbulence onset may be based on individual intervals 86, or based on more than two intervals 86. In an embodiment, turbulence slope is defined as the maximum positive slope of a regression line assessed over any sequence of five (5) subsequent sinus-rhythm intervals 86 within the first fifteen (15) sinus-rhythm intervals 86 after a premature ventricular contraction. In various alternative embodiments, the possible sample set of intervals 86 after a premature ventricular contraction may be as few as two and as many as thirty, while the regression line may be based on a sequence of as few as two (2) subsequent sinus-rhythm intervals 86 and as many intervals 86 as the size of the possible sample set.

In various embodiments, a dynamic change of heart rate turbulence (424) may be utilized to supplement or replace the instantaneous measurement of heart rate turbulence (424) described above. In certain embodiments, a change in turbulence slope of the heart rate turbulence marker (424) of less than two (2) milliseconds per R-R interval 86 may be factored in as a separate autonomics marker (408) in addition to heart rate turbulence marker (424) itself. Such a dynamic change turbulence slope marker may be modified by incorporating additional physiological conditions. In an alternative embodiment, a change in turbulence slope of the heart rate turbulence marker (424) of greater than two-and-a-half (2.5) milliseconds over one R-R interval 86 within four (4) weeks or six (6) weeks of a myocardial infarction may provide relatively little independent indication of patient risk. However, a dynamic change in turbulence slope of the heart rate turbulence marker (424) of less than or equal to two-and-a-half (2.5) milliseconds per R-R interval 86 during a period of remodeling of the heart following a myocardial infarction, in various embodiments four (4) and six (6) weeks, may be relatively strongly independently indicative of patient risk, in various embodiments from approximately three-and-a-half (3.5) to approximately nine (9) times as indicative as static, i.e., a measurement taken at a discrete point in time, indications of turbulence slope, thereby evincing a dynamic change in heart rate turbulence (424).

In an embodiment, a dynamic change of heart rate turbulence slope post acute myocardial infarction in patients with an ejection fraction of less than forty (40) percent may be incorporated as being indicative of increased likelihood of arrhythmia if the dynamic change in turbulence slope of the heart rate turbulence marker (424) is less than two (2) milliseconds per R-R interval 86. It is to be recognized and understood that other limits, ranges and variables may be effective in assessing the likelihood of a future arrythmia.

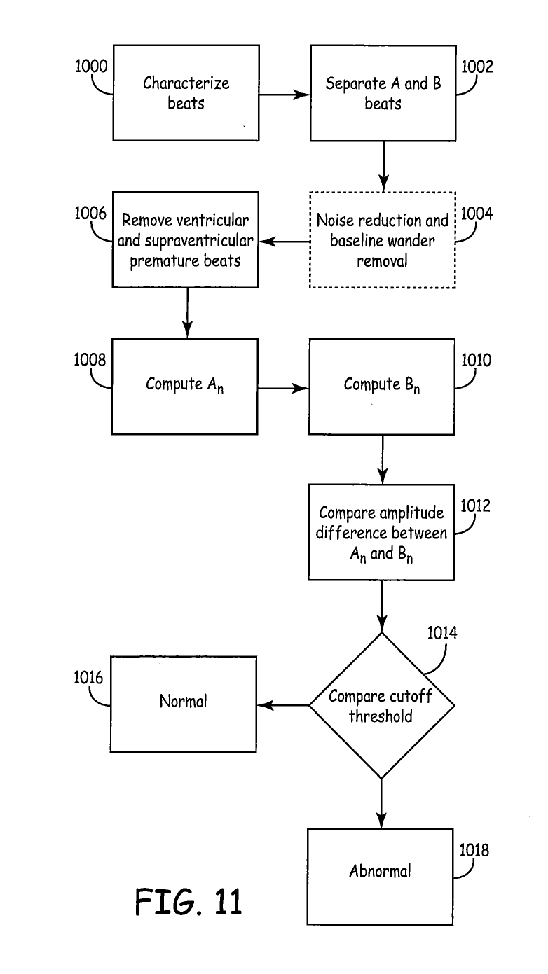

In an embodiment, if a heart rate turbulence condition is detected, an additional marker may be obtained relating to T-wave alternans. In particular, when heart 12 shows heart rate turbulence (424), T-wave alternans may be assessed according to the T-wave alternans analysis of FIGS. 10 and 11 below. Such a marker may be an additional marker relating to substrate category (406). Alternatively, such a marker may be an additional marker for autonomics category (408). Additional markers which are taken on the basis of two additional markers are contemplated. Additional markers may be obtained on the basis of timing relative to events. In an embodiment, markers may be obtained following an occurrence of a premature ventricular contraction.

Deceleration capacity (426) reflects a baseline autonomic tone and deceleration related changes in heart rate variability. Deceleration capacity, which reflects baseline vagal autonomic tone, may be contrasted to heart rate turbulence which reflects the autonomic reflex to perturbation in cardiac function. Deceleration capacity may provide a noninvasive means to assess the deceleration related changes in heart rate thereby reflecting vagal control, and may be easier and less traumatic to accomplish than via invasive procedures.

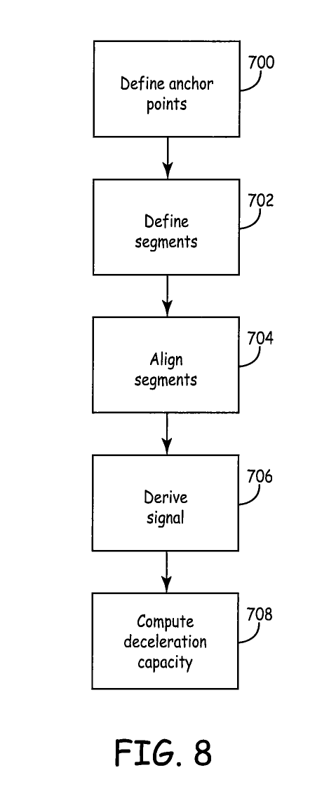

Deceleration capacity is based on the phase rectified signal averaging (PRSA) method.² The computational steps are illustrated in FIGS. 7a-7c and summarized in the flowchart of FIG. 8. Anchor points 88 are defined (700) as intervals 86 that are longer than an immediately preceding interval 86, illustrated as black circles in FIG. 7b. Next, segments 90 around anchor points 88 are defined (702). All segments have the same length and are chosen so as to resolve the lowest frequency in heart rate changes. Segments 90 are then aligned (704) around anchor points 88. Phase rectified signal averaging signal 92 is derived (706) by ensemble averaging of all of segments 90. Deceleration capacity is computed (708) according to the equation: ² Bauer et al, “Deceleration capacity of heart rate as a predictor of mortality after myocardial infarction: cohort study”, The Lancet, vol 367, May 20, 2006

DC(AC)=[X(0)+X(1)−X(−1)−X(−2)]/4 Equation 1

According to Equation 1, X(0) is anchor 88 about which the deceleration capacity is measured, X(1) is anchor 88 immediately following anchor 88 X(0), and X(−1) and X(−2) are anchors 88 immediately preceding anchor 88 X(0).

In various embodiments, the dynamic change of deceleration capacity (424) may be evaluated as a separate marker. The dynamic change of deceleration capacity may be indicative a likelihood of a future arrythmia or, rather, the dynamic change in the improvement of deceleration capacity may be indicative of a lower likelihood of a future arrythmia while the dynamic change in the deterioration of deceleration capacity or lack of change of deceleration capacity may be indicative of an increased likelihood, relatively, of a future arrythmia.

Examples of data related to arrhythmia burden which are monitored (426, FIG. 4) may include a number of premature ventricular contractions per hour (428), a duration and/or rate of non-sustained ventricular tachycardia (430), a non-sustained ventricular tachycardia heart rate (432), an absolute number of premature atrial contractions over a given time period (434), measurements of a frequency of premature ventricular contractions (PVC) over a given time period (436), such as a number of premature ventricular contractions per hour, and an atrial fibrillation burden (438). In general, as known in the art, atrial fibrillation burden (438) represents a frequency of occurrence of an atrial fibrillation rhythm as detected by implanted device 30 over an extended period of time. For instance, one can assess how often a patient's heart rhythm was in atrial fibrillation over a twenty-four (24) hour period, a one-to-four week period, a one-to-twelve month period, or over multiple years, thereby evincing a dynamic change in atrial fibrillation burden.

As indicated above, it is understood that various markers known in the art or currently under research and development efforts are or may be effective for use in combination with, addition to or in supplement for the makers detailed above. For instance, markers pertaining to a relationship between nonsustained ventricular tachycardia after non-ST-elevation acute coronary syndrome, ejection fraction in relation to heart rate turbulence and/or T-wave alternans, and a number of intervening beats between premature ventricular contractions may be utilized in accordance with the methodology described herein. Moreover, such enumerated additional markers do not limit the scope for further additional markers to be utilized in a manner consistent with this disclosure.

In an embodiment, after the cardiac data is collected according to FIG. 4, the risk stratification algorithm utilizes the cardiac data to obtain risk stratification. In an embodiment, illustrated in the flowchart of FIG. 9, the risk stratification algorithm factors in (800) a heart rate turbulence (422, FIG. 4) onset and evaluates (802) whether the turbulence onset is less than a threshold, and therefore normal, and evaluates (804) whether the turbulence slope is greater than a threshold, and therefore normal. In an embodiment, the threshold for turbulence onset is zero and the turbulence slope threshold is 2.5 milliseconds per interval 86. In alternative embodiments, the turbulence slope threshold may be less than 2.5 milliseconds to provide relatively more relaxed requirements for normalcy, and greater than 2.5 milliseconds if the requirements for normalcy may be relatively more stringent. In an embodiment, turbulence slope is the maximum slope of the regression line that fits five (5) intervals 86 during up to thirty (30) beats following a premature ventricular contraction. In alternative embodiments, the regression line may fit more or fewer intervals 86 during more or fewer beats following a premature ventricular contraction. Factoring in both turbulence onset and turbulence slope, the risk stratification algorithm may determine (806) a risk of sudden cardiac death. If both the turbulence onset and the turbulence slope are normal, i.e., the turbulence onset is less than the threshold and the turbulence slope is greater than the threshold, the risk may be identified as low (808). If one but not both of turbulence onset and turbulence slope is normal, i.e., one but not the other is abnormal, then the risk may be identified as moderate (810). If both turbulence onset and turbulence slope are abnormal then the risk may be identified as high (812).

In various embodiments, the risk stratification algorithm considers the T-wave alternans marker (412, FIG. 4). In brief, and in an embodiment, the T-wave alternans marker (412) determines the maximum difference in the ST-T windows between two modified moving average templates constructed from alternate beats. A weighted moving average is applied to limit the effect of artifacts and the contributions of single beats. The weighted moving average algorithm differs from a spectral domain microvolt T-wave alternans method known in the art in that it does not require data to be stationary, implying fixed at a specific heart rate.

In an embodiment, Tₚₑₐₖ 80 of the consecutive T-waves 74 is measured and subtracted from one another, with the absolute value of the difference compared against a cutoff threshold. In an alternative embodiment, peak-to-peak amplitude for each T-wave is measured and subtracted. In various embodiments, the cutoff threshold is selected over a range from twenty (20) microvolts to fifty (50) microvolts. In various embodiments, the cutoff threshold is selected from the range of thirty-one (31) microvolts to thirty-seven (37) microvolts. In an embodiment, the cutoff threshold is thirty-four (34) microvolts. If the absolute value of the difference in measured Tₚₑₐₖ values following premature ventricular contraction 86 is less than the threshold, compensatory pause 90 and, in an embodiment, an abnormal autonomic reflex, then patient 10 may be identified as not having significant T-wave alternans and, as a result, as being at higher risk of future arrhythmia.

In such embodiments, and as will be described in detail below, T-wave alternans (412) may be measured at various times and recorded in memory 52. The T-wave alternans (412) measurements may be compared to identify dynamic changes in T-waves for patient 10. In various embodiments, a difference in consecutive T-wave alternans (412) measurements may be subtracted in order to show a difference between T-wave alternans (412) measurements over time. In an embodiment, T-wave alternans (412) are measured at six hour intervals. In an alternative embodiment, T-wave alternans (412) are measured daily. In alternative embodiments, T-wave alternans (412) are measured more or less frequently. In various such embodiments, dynamic changes in T-wave alternans (412) may be normalized by dividing the difference in measured T-wave alternans (412) by the duration in time between the measurements, providing a rate of change of T-wave alternans. Alternatively, the dynamic change of T-wave alternans may be evaluated by comparing the difference between successive T-wave alternans (412) measurements against an absolute threshold.

The dynamic change of T-wave alternans (412) may then be compared against a threshold. In various such embodiments, if the dynamic change in T-wave alternans (412) is less than or equal to negative-two (2) microvolts per twenty-four (24) hours then patient 10 is deemed as being at low risk, relatively, of future arrhythmia irrespective of patient's 10 instantaneous T-wave alternans (412) measurement. Alternatively, if the instantaneous T-wave alternans (412) measurement indicate a level sufficiently high, in an embodiment greater than or equal to sixty (60) microvolts, patient 10 may be evaluated as being at higher risk, relatively, of future arrhythmia irrespective of a dynamic change in T-wave alternans. Further alternatively, instantaneous and dynamic change measurements may be factored jointly in determining risk. For instance, in an embodiment, if patient 10 has an instantaneous T-wave alternans (412) measurement of forty-five (45) microvolts and a dynamic change of negative one (1) microvolt per twenty-four (24) hours, patient 10 may be evaluated as not being at increased risk of future arrhythmia, thereby evincing a dynamic change in T-wave alternans (412).

In alternative embodiments, the T-wave alternans (412) metric utilizes the modified moving average analysis as understood in the art and as described by Nearing, Bruce D. and Verrier, Richard L., in “Modified moving average analysis of T-wave alternans to predict ventricular fibrillation with high accuracy”, J. Appl Physiol 92: 541-549, 2002, which is incorporated herein in its entirety. FIG. 10 illustrates the modified moving average beat analysis method, which is further shown in the flowchart of FIG. 11. Heart beats are alternately characterized (1000) as A and B beats. In an embodiment, the signal is optionally subjected to noise reduction and baseline wander removal (1002), then the A and B beats are separated (1004). Ventricular and supraventricular premature beats are removed (1006) on the basis of a comparison of R-R intervals 86 with an R-R median at the first A beat A₁ if the R-R interval 86 is, in an embodiment, less than eighty (80) percent of the R-R median or greater than one hundred twenty (120) percent of the R-R median. A computed Aₙ beat is equal (1008) to the preceding computed Aₙ₋₁ plus the change in the A waves. The change is determined by a weighted difference between the current A beat and the preceding computed Aₙ₋₁. A computed Bₙ beat is computed (1010) in the same way. The alternans measurement is obtained by comparing (1012) the difference in amplitude between the computed Aₙ beat and the computed Bₙ beat. In various embodiments, the number of heart beats utilized may be selectable. In an embodiment, the number of heart beats utilized may be sixteen, organized into eight consecutive A-B pairs.

In various embodiments, if the median of interval 86 is less than approximately four hundred (400) milliseconds or greater than approximately two thousand (2000) milliseconds, the T-wave alternans analysis may be discarded or restarted. Additionally, in an embodiment, if three (3) consecutive beats are premature ventricular contractions 85, the T-wave alternans analysis may be discarded or restarted. Additionally, in an embodiment, if two (2) consecutive beats are greater than approximately two thousand (2000) milliseconds, the T-wave alternans analysis may be discarded or restarted.

In such embodiments, a cutoff threshold may be established and compared (1014) against the alternans measurement. In various embodiments, the cutoff threshold is a predetermined value. In an embodiment, the cutoff threshold is forty (40) microvolts. If the modified moving average is less than the cutoff then T-wave alternans (412) are normal (1016). If the modified moving average is greater than or equal to the cutoff then the T-wave alternans (412) are abnormal (1018). In various alternative embodiments, cutoff thresholds may be selected based on a presence of other metrics which tend to suggest a patient is at a relatively high risk or a relatively low risk of a cardiac condition. In an embodiment, a cutoff threshold of twenty (20) microvolts is applied to patients who have at least one additional marker which indicates the patient is at risk of a cardiac condition. In an embodiment, a cutoff threshold of sixty (60) microvolts is applied to patients who have no additional markers which indicate the patient is at risk of a cardiac condition.

In various embodiments, a modified moving average analysis as applied to T-wave alternans (412) above may be applied to other metrics. Application of a modified moving average may create alternate markers. In an embodiment, for instance, one alternate marker which may be utilized is to apply a modified moving average analysis to a maximum heart rate of patient 10 over each of a number of predetermined and predefined periods. For instance, in an embodiment, a maximum heart rate on each of a predetermined number of days may be subjected to modified moving average analysis according to FIG. 10.

Continuous monitoring of T-wave alternans (412) according to modified moving average analysis using minimally invasive devices, such as implantable device 10, offers the potential for (a) assessing a patient's “repolarization burden” over time, thereby circumventing the disadvantage of a single point in time monitoring, (b) tracking myocardial substrate remodeling after an index event, and (c) monitoring an effect of therapy delivered to patient 10 and, in particular, heart 12. In various embodiments, the cardiac signals generated by heart 12 may be manipulated to facilitate analysis. In various embodiments, the cardiac signal is downsampled to 128 Hertz or to 256 Hertz, subjected to a bandpass filter of 0.5 Hertz-95.0 Hertz and scaled to 0.3662 μV per bit. In such an embodiment, a crescendo in T-wave alternan amplitude may be predictive of spontaneous ventricular tachycardia resulting in a relatively significant rise (p<0.05) in modified moving average values at zero to thirty (30) minutes prior to ventricular tachycardia, relative to a baseline value taken forty-five (45) to sixty (60) minutes prior to an onset of ventricular tachycardia. In other words, an increase in the modified moving average relative to a baseline may be predictive of ventricular tachycardia approximately thirty (30) to forty-five minutes after the increase begins.

In various embodiments, a dynamic change of T-wave alternans (412) which correspond to a baroreflex response of patient 10 may be analyzed. In such an embodiment, autonomic markers (408) may be utilized to identify an abnormal baroreflex response in patient 10 on the basis of an abnormal autonomic condition as described above and below. In various embodiments, dynamic changes to T-wave alternans (412) may be measured during circumstances in which at least one autonomic marker (408) is outside of a corresponding range, thereby indicating a condition of T-wave alternans (412) induced by a baroreflex response. Such a measurement may be separately indicative of a higher risk, relatively, of future arrhythmia compared with other measurements of dynamic change in T-wave alternans (412).

In an embodiment, a maximum modified moving average T-wave alternans measurement for an observation period, in an embodiment six (6) minutes long, may be added to other maximum modified moving average T-wave alternans measurement to create sums for predetermined extended periods. In an embodiment, the predetermined extended period is one day. In an embodiment, if the six (6) minute observation period falls within 0400 hours to 1200 hours and 1600 hours to 1800 hours, the maximum modified moving average T-wave alternans within the observation period may be added to the maximum value sum for an applicable two (2) hour period, such as 0400 hours to 0600 hours, 0600 hours to 0800 hours, 0800 hours to 1000 hours, 1000 hours to 1200 hours or 1600 hours to 1800 hours. In further embodiments, the modified moving average T-wave alternans value that uses the shortest R-R interval 86 median as its comparison value may be stored each day, along with the corresponding R-R interval 86 median, thereby evincing a dynamic change in T-wave alternans (412) corresponding to a baroreflex in patient 10.

In an embodiment, T-wave alternans may also be assessed on the first eight (8) beat pairs following the premature ventricular contraction 85 of heart rate turbulence analysis (424). The modified moving average T-wave alternans values associated with all heart rate turbulence-segments for a day may be summed. The modified moving average T-wave alternans value and associated heart rate turbulence-segment data may be accumulated when the modified moving average T-wave alternans value is available.

In various embodiments, the risk stratification algorithm considers the number of premature ventricular contractions per hour (428). In such embodiments, the number of premature ventricular contractions per hour is compared against a cutoff threshold. In an embodiment, the cutoff threshold is ten (10) premature ventricular contractions per hour. In alternative embodiments, the cutoff threshold may be more or fewer than ten (10) premature ventricular contractions. If the number of premature ventricular contractions per hour are greater than or equal to the cutoff then the patient may be identified as being at high risk of arrhythmias. If the number of premature ventricular contractions are less than the cutoff then the patient may be identified as being at low risk of arrhythmias.

In alternative embodiments, time periods of more or less than one hour may be utilized. In an embodiment, the time periods may be selectable in increments of one minute. In such an embodiment, the cutoff threshold may be varied to compensate for the changed time period. In an embodiment, the cutoff threshold is changed proportional to the change in the time period. In various embodiments, the cutoff threshold is maintained as an integer.

In addition, as shown in FIG. 4, genetic information (404) relating to the patient and to clinical demographic information such as, but not limited to, age, ejection fraction, history of atrial fibrillation, and conduction disorders such as left bundle branch block and/or right bundle branch block may be incorporated as genetic and/or clinical data. Such data may be converted into qualitative or quantitative scores and applied like measured markers.

It is known in the art that patients with a relatively low ejection fraction may be indicated as having or being susceptible to heart failure. Factoring in the ejection fraction of the patient may impact the assessed risk the patient carries. In particular, a patient with a low ejection fraction may be indicated as being at risk of sudden cardiac death and/or heart failure. In various embodiments, the risk stratification algorithm factors in whether the patient's ejection fraction is less than or equal to thirty-five (35) percent. If the ejection fraction is less than or equal to thirty-five (35) percent, patient 10 may be evaluated as being at high risk of sudden cardiac death. If the ejection fraction is greater than thirty-five (35) percent, the patient may be at a low risk of sudden cardiac death. Additional thresholds may be utilized based on well-known standards for evaluating other cardiac risks based on ejection fraction, such as heart failure.

As discussed above, ejection fraction may be a factor or marker which is used in conjunction with other markers. In other words, other makers may be of greater significance in view of a low ejection fraction than may be the case absent a low ejection fraction. Ejection fraction may, in various embodiments, be a marker in its own right and independent of other markers. In such embodiments, ejection fraction may be a static marker, in an embodiment being indicative of risk if the ejection fraction is less than or equal to thirty (30) percent four (4) weeks after a myocardial infarction. Alternatively, risk may be indicated if the ejection fraction is less than or equal to thirty (30) percent six (6) weeks after a myocardial infarction. In various alternative embodiments, ejection fraction is a dynamic marker, wherein if a patient has an ejection fraction of less than or equal to thirty (30) percent after a myocardial infarction and the ejection fraction does not increase during a remodeling period of the heart following myocardial infarction, in various embodiments four (4) weeks and six (6) weeks. In various embodiments, the dynamic ejection fraction marker indicating a lack of recovery of the ejection fraction may be fractionally more predictive of patient risk than static measurements of ejection fraction. In certain embodiments, the dynamic ejection fraction marker is from approximately eighteen (18) percent to approximately twenty-seven (27) percent more predictive of patient risk than static markers, thereby evincing a dynamic change in ejection fraction.

The above particular cases are illustrative of how data relating to risk stratification may be analyzed. Any of the factors shown in FIG. 4, as well as any other factors well known in the art, may be utilized in the risk stratification algorithm according to judgments of one skilled in the art as to what would constitute normal or abnormal states for such factors according to known standards.

While individual tests or measurements, such as those described above, may provide some indication, i.e., stratification, of risk of experiencing ventricular or atrial arrhythmias such as fast ventricular tachycardia or ventricular fibrillation, results from a plurality of markers may improve stratification for the likelihood of experiencing ventricular arrhythmias such as fast ventricular tachycardia and ventricular fibrillation. Additionally, atrial arrhythmias may similarly be detected.

In various embodiments, the results of each marker may be accorded a score indicative of the likelihood of a patient to experience ventricular or atrial arrhythmias such as fast ventricular tachycardia and/or ventricular fibrillation. Such results may be expressed either qualitatively or quantitatively.

A quantitative expression may be, for example, a numerical score accorded to the result. As an example, a numerical score greater than a predetermined threshold may be indicative of a relatively greater likelihood that the patient will experience ventricular or atrial arrhythmias such as fast ventricular tachycardia or ventricular fibrillation. Similarly, a numerical score smaller than a predetermined threshold may be indicative of a relatively lesser likelihood that the patient will experience ventricular or atrial arrhythmias such as fast ventricular tachycardia or ventricular fibrillation. In various embodiments, alternative scoring techniques may be utilized. For instance, relating to the premature ventricular contractions per hour marker (428), the actual number of premature ventricular contractions per hour may be the quantitative expression for the premature ventricular contractions per hour marker (428). Such values may then be weighted to bring the quantitative analysis in line with other markers. By contrast, in various embodiments, the quantitative evaluation for each marker may be obtained by setting multiple related thresholds for each marker and assigning a numerical value for each threshold crossed. Thus, by way of illustration, for T-wave alternans, if the modified moving average is less than twenty (20) microvolts, a qualitative value of zero (0) may be set; if the modified moving average is greater than twenty (20) microvolts but less than thirty (30) microvolts, a qualitative value of one (1) may be set; if the modified moving average is greater than thirty (30) microvolts but less than forty (40) microvolts a qualitative value of two (2) may be set; if the modified moving average is greater than forty (40) microvolts a qualitative value of three (3) may be set. Similar data may be obtained for each marker, and the qualitative values may be included in the quantitative evaluation for each category.

Quantitative values for additional markers may be selected based on similar applications to expected results and commonly known variations from typical results.

The quantitative scores from each measurement technique may be combined to obtain a quantitative or qualitative score representative of a likelihood that a patient will experience ventricular or atrial arrhythmias such as fast ventricular tachycardia or ventricular fibrillation. For example, the numerical score from each measurement may be combined by adding the scores together. In various embodiments, weighting factors may be applied to various markers to create greater emphasis on certain markers and lesser emphasis on other markers.

In an embodiment, autonomic markers may be relatively less predictive of future arrhythmia when an ejection fraction of patient 10 is less than or equal to thirty-five (35) percent. In various such embodiments, autonomic markers (408) may be assigned a relatively lower weight when the ejection fraction is less than or equal to thirty-five (35) percent. In one embodiment, autonomic markers (408) may be assigned a weight of 0.2, substrate markers (406) may be assigned a weight of 0.2, arrhythmia burden markers (410) may be assigned a weight of 0.3 and genetic markers (404) may be assigned a weight of 0.3.

In additional embodiments, patients with high ejection fractions but who have suffered from a previous acute myocardial infarction, autonomic markers (408) may have a relatively significant predictive effect. In one embodiment, autonomic markers (408) may be assigned a weight of 0.3, substrate markers (406) may be assigned a weight of 0.3, arrhythmia burden markers (410) may be assigned a weight of 0.3 and genetic markers (404) may be assigned a weight of 0.1. In various alternative embodiments classes of markers (406), (408), (410) are not assigned weights, but rather particular markers are assigned weights. In one such embodiment, in which patient 10 has an ejection fraction of greater than thirty-five (35) percent and who had suffered a previous acute myocardial infarction, heart rate turbulence (424) has a weight of 0.3, T-wave alternans (412) has a weight of 0.3, premature ventricular contractions per hour (428) has a weight of 0.2, non-sustained ventricular tachycardia rate (436) has a weight of 0.1 and genetics (404) has a weight of 0.1.

In various embodiments, the weighting factors may be dynamic, changing based on particular circumstances of patient 10. In particular, each of markers, i.e., biological parameters, may be dynamically weighted based on another one of the markers or plurality of biological parameters of the patient. In an exemplary embodiment, heart 12 being in atrial fibrillation may cause certain markers to be weighted relatively more heavily than others. For instance, detecting atrial fibrillation may result in an increased weighting, e.g., a doubling of the effect, of QRS duration (414) and QRST integral (416). A detection or incorporation of a genetic mutation into genetic markers (404) which indicate a propensity for atrial fibrillation may result in a lower weight for various arrhythmia burden markers (410) relating to atrial fibrillation as it is already known that such a patient 10 is at risk of atrial fibrillation. In such circumstances, autonomic markers (408) and substrate markers (406) may be given relatively higher weights.

A detection or incorporation of a genetic marker such as a conduction disorder may result in changes in weighting of all markers of substrate group (406). In alternative embodiments, only some markers of substrate group (406) are weighted differently. In various embodiments, all markers of substrate group (406) may be altered equally. In alternative embodiments, markers of substrate group (406) may be altered variably based on an actual type of conduction disorder detected or entered. For instance, a right bundle branch block may result in a heavier weighting for QRS duration (414) and QRST integral (416) markers relative to the rest of markers of substrate group (406), though the rest of the markers of substrate group (406) may have their weighting changed. Similarly, if patient 10 suffered from left or right bundle branch block, T-wave alternans (412), QRST integral (416), QT variability index (420) and autonomics markers (408) generally may be more heavily weighted while QRS duration (414) may be less heavily weighted owing to prolonged QRS duration being expected to be experienced in a patient who has suffered right or left bundle branch block.

In certain cases, trends in changes in measured or computed markers may be predictive of future arrhythmias. By tracking and evaluating dynamic changes in markers, risk assessments may be modified. In various embodiments, the dynamic changes in markers may themselves be compared against predetermined thresholds and risk assessments may be made on the basis of the comparison. In that way, the dynamic change data may function as an additional marker and weighted and incorporated into risk assessment analyses accordingly. Alternatively, dynamic changes in markers may serve as “triggers” for further analysis. In such embodiments, when a dynamic change in one marker indicates that a patient is at risk, additional marker sensing and processing may be performed to more fully assess the risk of patient 10 suffering from a future arrhythmia.

In alternative embodiments, quantitative scores may be developed based on multiplying the scores of individual markers together. Similarly with the quantitative scoring utilizing addition, various forms of weighting may be applied to the individual markers.

In contrast to quantitative results, qualitative results may be expressed, not as numerical values, but rather as more granular assessments of risk. In various embodiments, the quantitative analysis may be “high” or “low”, or may be “high”, “middle” or “low”, for example. Other qualitative expressions are also contemplated. Qualitative results from each measurement technique may be combined to obtain a qualitative score representative of an overall likelihood that a patient may experience ventricular or atrial arrhythmias such as fast ventricular tachycardia or ventricular fibrillation.

In additional embodiments, either quantitative or qualitative scores may be combined together, for instance by cross-assigning qualitative or quantitative scores, as the case may be, to respective data. For instance, a quantitative score of from “0” to “3” may correspond to a qualitative score of “low”, while a qualitative score of “low” may correspond to a quantitative score of “1”.

In various embodiments, other measurement techniques, other than those described herein, may be utilized that may be, at least in part, indicative of establishing a degree of risk that a patient will experience ventricular or atrial arrhythmias such as fast ventricular tachycardia or ventricular fibrillation. In various embodiments, a plurality of measurement techniques may be used or a particular number of measurement techniques in excess of two, for example, three or four, may be used. In an embodiment, the particular measurement techniques employed may be chosen from among those available.

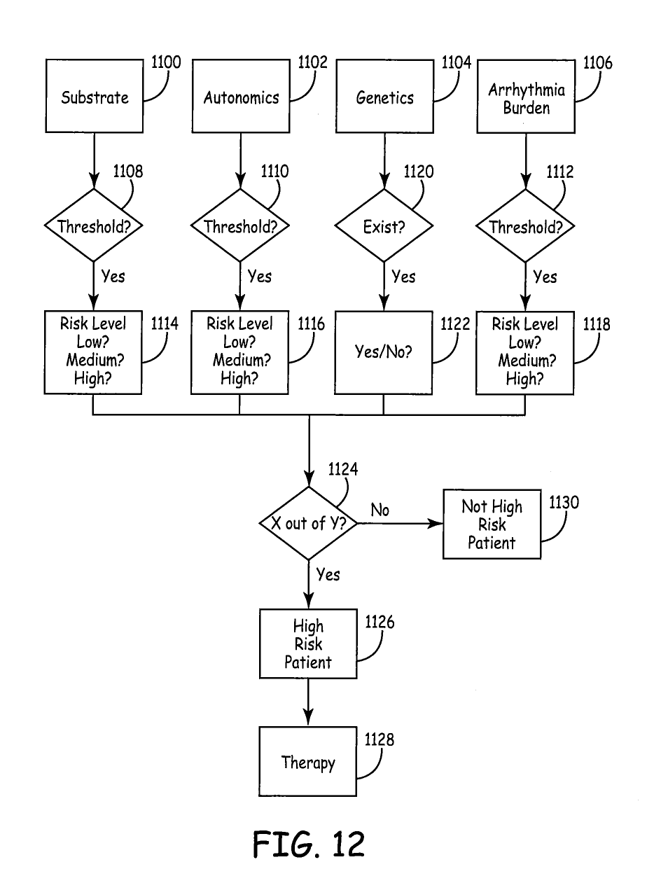

FIG. 12 is a flowchart showing one embodiment of the risk stratification algorithm which utilizes qualitative assessments of each category. In it, various data related to the cardiac substrate (1100, corresponding to 406, FIG. 4), cardiac autonomics (1102, corresponding to 418, FIG. 4), genetics (1104, corresponding to 404, FIG. 4) and arrhythmia burden (1106, corresponding to 410, FIG. 4) are collected. As illustrated, data related to all of four categories are collected. In alternative embodiments, particularly where such data is not available or is not readily available, data related to only some of the categories are collected. In various embodiments, for each category for which data is provided, at least one marker is utilized. In alternative embodiments, at least two markers are utilized in at least one category. For each of the categories for which data is utilized, the data are compared (1108, 1110, 1112) against thresholds or cutoffs as described above, and individual qualitative risk assessments for each category are obtained (1114, 1116, 1118). In various embodiments, at least one or more of the markers utilized include markers relating to dynamic change of physiological parameters, as described above, for instance, with regard to T-wave alternans (412), heart rate turbulence (424) and deceleration capacity (426), though not limited to such markers. In such embodiments, the dynamic change marker or markers function as markers along with markers associated with instant measurements. As illustrated, the qualitative risk for each category is assessed as being “low”, “medium” or “high”. In the case of genetic information, an assessment may not be against a threshold or cutoff, but rather a binary assessment (1120) of whether or not a particular risk factor exists and a qualitative risk assessment obtained (1122) for genetic information. As illustrated, the qualitative risk for genetics is either “yes” or “no”, according to the individual risk factors.

Once each of the categories which include data is assessed for risk factors, the individual risk factors are combined (1124) or pooled to obtain a general assessment of patient risk for sudden cardiac death. In particular, if a particular number of categories X out of the total number of categories assessed Y indicate risk of sudden cardiac death, the patient is evaluated as being at high risk (1126). As illustrated, where the categories are assessed as having “low”, “medium” and “high” risk, if four categories have data, then the patient may be evaluated as being at high risk if at least two categories have high risk, or, in the case of genetics, a “yes” result, at least one category has high risk and at least two categories have medium risk, or if all four categories have medium risk. If three categories have data, then the patient may be evaluated as being at high risk if at least two categories have high risk, at least one category has medium risk and one category has high risk, or if all three categories have medium risk. Alternative relationships are contemplated. If the requirement for high risk is met, patient 10 may be treated (1128) with therapy. If the requirements for high risk are not met (1130), no further action may be taken, or the patient may be monitored in the future.

Alternatively, markers indicative of dynamic change may dominate or be dispositive over markers related to instantaneous measurements. Further, alternatively, as described above, markers indicative of dynamic change may be dispositive when markers indicative of instantaneous measurements meet certain requirements and not dispositive when the instantaneous markers do not meet the requirements. For instance, in an embodiment, if T-wave alternans (412) measured instantaneously are above a predetermined threshold, such as sixty (60) microvolts, the dynamic change in T-wave alternans is not dispositive, while if the instantaneous measurement in T-wave alternans (412) is less than or equal to sixty (60) microvolts then the dynamic change T-wave alternans (412) may be dispositive over the T-wave alternans (412) instantaneous measurement.

Where each category has a risk assessment of either “low” or “high”, then the number of “high” results are simply compared. In an embodiment, if at least two out of four categories show a “high” risk or, in the case of genetics a “yes” result, then the patient is assessed as having high risk of sudden cardiac death. In an embodiment where only three categories are assessed, if two out of three categories show “high” risk then the patient is assessed as having high risk of sudden cardiac death. In embodiments where two categories have data, the patient may be evaluated as being at high risk of sudden cardiac death if one category has “high” risk. Alternative relationships are contemplated.

Based on the assessment of the qualitative evaluations of each category, patient 10 may be indicated for an implantable medical device which provides therapy suitable to treat the condition to which the risk stratification algorithm indicates the patient may be susceptible. Such implantable medical devices include pacemakers and cardioverter/defibrillators, and may be further configured to treat conditions such as congestive heart failure and the like.

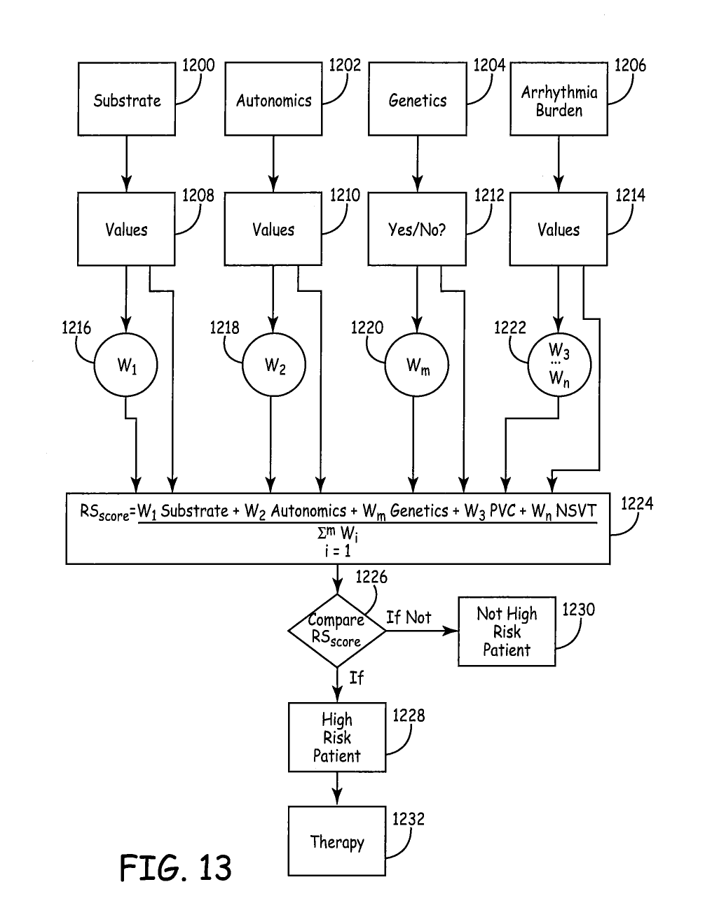

FIG. 13 is a flowchart of an alternative embodiment of risk assessment algorithm which utilizes a quantitative assessment of each category. Similarly to FIG. 12, various data related to the cardiac substrate (1200, corresponding to 406, FIG. 4), cardiac autonomics (1202, corresponding to 418, FIG. 4), genetics (1204, corresponding to 404, FIG. 4) and arrhythmia burden (1206, corresponding to 426, FIG. 4) are collected. Quantitative evaluations of the data of each category are obtained (1208, 1210, 1212, 1214), and weighted (1216, 1218, 1220, 1222) for the risk stratification algorithm. As shown, each quantitative evaluation for each marker is weighted by a predefined weight W for each category with weight W₁ for the substrate category, W₂ for autonomic, Wₘ for genetics and multiple W₃-Wₙ weights for individual markers in the arrhythmia burden category. The quantitative evaluation for each category is utilized by the risk stratification algorithm to obtain (1224) a score RS_{score}, obtained, as illustrated, by summing all of the available quantitative marker values weighted by their corresponding weights W₁, W₂, W₃, Wₙ and Wₘ available and dividing that by the number of quantitative evaluations provided. The score RS_{score} is then compared (1226) against a threshold in order to determine whether the patient is at high risk (1228) or not (1230). If patient 10 is considered to be high risk, therapy may be delivered (1232).

In various embodiments, the sum of the values of the weights is one (1). In various such embodiments, RS_{score} is normalized so that it is between zero (0) and (1), and a resultant RS_{score} of less than 0.25 indicates low risk, 0.25 to 0.75 indicates a moderate risk and greater than 0.75 indicates a high risk.

In various alternative embodiments, dynamic changes in markers may be weighted and applied along with markers corresponding to instantaneous measurements. In certain such embodiments, markers indicative of instantaneous measurements may be weighted relatively lower than markers indicative of dynamic change. In one embodiment, markers indicative of instantaneous measurements are not utilized if a marker indicative of dynamic change for the same physiological parameter is available. For instance, if a dynamic change marker for T-wave alternans (412) is available then a marker for an instantaneous measurement for T-wave alternans (412) would not be utilized.

In an alternative embodiment, quantitative values for each marker may be utilized directly by the risk stratification algorithm without consideration within each category. In such an embodiment, the quantitative values for each marker may be summed together and divided by the total number of markers to obtain the RS_{score} value. In the embodiments described, on the basis of the RS_{score} the patient may be indicated for implantation of an implantable medical device as described above.

In various additional embodiments, the risk stratification algorithm may provide more than a binary assessment of risk, i.e., a quantitative risk assessment. In such embodiments, a relatively high numeric assessment of risk may indicate that the patient may benefit from the implantation of an implantable device while a very low numeric assessment of risk may indicate that the patient is in no further need of treatment or monitoring. Medium levels of assessed risk, however, may suggest that the patient is in little need of additional therapy but should be monitored. Further medium levels of assessed risk may indicate that the patient may benefit from preemptive drug therapies, but may not yet be indicated for an implantable device. Varying assessments of risk may provide varying conclusions for what treatment is provided, and such assessments and treatments may be determined on case-by-case bases.

In an embodiment relating to FIGS. 12 and 13, markers utilized include heart rate T-wave alternans (412), turbulence (424), premature ventricular contractions per hour (428) and a modified moving average of a maximum daily heart rate as described above.

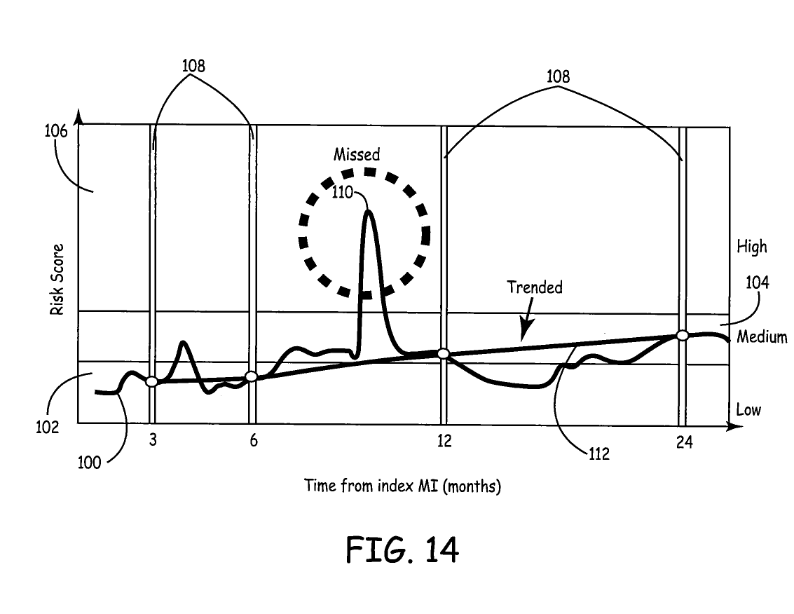

FIG. 14 illustrates an example of a particular utility of implantable device 30, which may monitor patient 10 continuously for as briefly as forty-eight (48) hours and more than twenty-four (24) months, in contrast with a conventional monitoring device, such as a Holter monitor, which typically monitors for a matter of hours or days. Risk line 100 represents a quantified index of risk of sudden cardiac death in patient 10 compared against horizontal bands 102, 104, 106 representing low, medium and high risk, respectively. The vertical lines 108 represent periods in which cardiac data is monitored variably by a Holter monitor and in a clinician's office. As illustrated, patient 10 experienced a spike 110 in risk line 100 which indicated a high ongoing risk of sudden cardiac death, but because the Holter monitor was not operating and because patient 10 was not being analyzed in a doctor's office, the indication was missed. Under these circumstances, the patient may have provided an indication of risk, but the indication is missed, thereby leaving an at-risk patient not-indicated for implantation with a device which could save the patient's life in the event of sudden cardiac death. By contrast, the combination of Holter monitor and clinician office visit would merely provide trend line 112 indicating a medium level of risk, well below the actual risk noted by risk line 100.

In various embodiments, analysis may occur not continually but rather at appointed times during each day of an extended period of time. In various embodiments, measurements may be obtained during predetermined time periods during a day. In an embodiment, measurement windows may be established, such as two hours. The measurement windows may be assigned during a day as determined by a medical professional. Such assignments may be on the basis of patient need. For instance, in various embodiments, a medical professional may assign windows based on a time of day at which patient 10 wakes up in the morning and eats meals. In such an embodiment, two-hour windows may be assigned from 6:00 AM to 8:00 AM, 8:00 AM to 10:00 AM, 10:00 AM to 12:00 noon and 4:00 PM to 6:00 PM. Windows may be varied in duration, number per day and timing during a day. Further, such data windows may extend for more than one day, and may be assigned on weekly, monthly or yearly bases.

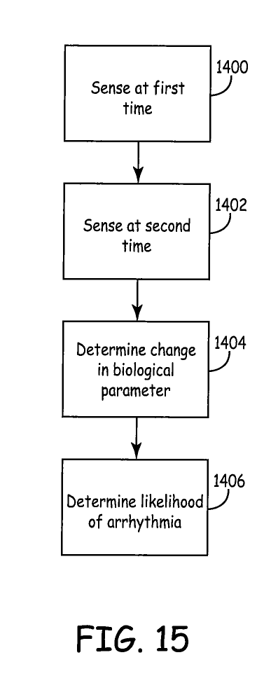

FIG. 15 is a flowchart for assessing a likelihood of a patient to experience a future cardiac arrhythmia utilizing a dynamic change in markers. In an embodiment, a change in a biological parameter is obtained by sensing a biological parameter of patient 10 with sensor 54 at a first time, then sensing the biological parameter of patient 10 with the sensor 54 at a second time, then determining a change of the biological parameter from the first time to the second time with implantable medical device 30. A biological parameter is measured (1400) at a first time using electrodes 32, 34 and sensor 54 of implantable device 30. In various embodiments, the biological parameter is one of substrate (406), autonomic (408) and arrhythmia burden (410) markers. Then the biological parameter is measured (1402) at a second time, different from the first time. Then a change in the biological parameter from the first time to the second time is determined (1404), thereby evincing a dynamic change in the markers measured. In various embodiments, the change is determined by processor 50 of implantable device 30. In alternative embodiments, a processor in an external or other device makes the determination. A likelihood of patient 10 experiencing a cardiac arrhythmia is then determined (1406) based, at least in part, on the change in the biological parameter.

In various embodiments, sensing (1400, 1402) occurs after patient 10 has suffered a myocardial infarction. In alternative embodiments, sensing (1400, 1402) occurs after a patient has been interacted with by a medical professional, such as having been hospitalized, had a medical checkup in a clinic, or other similar encounter whether in person or remotely. Such encounters may generally be categorized as having been “hospitalized”, as is known in the art. In various embodiments, the first time and the second time are separated by six hours and twenty-four hours. In addition, various other durations are applicable. Further, in various embodiments, the biological parameter is sensed (1400, 1402) as a maximum sensed value over a period of time corresponding to each of the first time and the second time, or as an average sensed value over the period of time corresponding to each of the first time and the second time. In various embodiments, the period of time is approximately two hours, as described with respect to FIG. 14. Alternative periods of time may also be utilized under various circumstances.

While various embodiments have been described using a dynamic marker and/or a dynamic marker in conjunction with a static marker (a marker measured at one point in time), it is to be recognized and understood that a plurality of dynamic markers may also be utilized in the determination of a likelihood of a future arrythmia. For example, two or more dynamic markers, perhaps the rate of change of T-wave alternans and a rate of deceleration capacity, could be used together, at least in part, to determine a likelihood of a future arrythmia. Likewise, two or more dynamic markers could be used in conjunction with one or more static markers to, at least in part, establish a likelihood of a future arrythmia.

Embodiments have been described in which a value of one or more markers could affect the weighting given to another marker in determining, at least in part, the likelihood of a future arrythmia. It is to be recognized and understood that one or more markers either affecting weighting or being affected by weighting could be dynamic markers.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A system for assessing a likelihood of a patient to experience a future cardiac arrhythmia, comprising:
a biological sensor configured to sense a biological parameter of said patient;
a processor operatively coupled to said biological sensor and configured to:
determine a dynamic change of said biological parameter; and
determine said likelihood of said patient experiencing a cardiac arrhythmia based, at least in part, on said dynamic change of said biological parameter.

2. The system of claim 1 wherein said processor determines said dynamic change based, at least in part, on said dynamic change of said biological parameter from a first time to a second time.

3. The system of claim 2 wherein said processor determines said dynamic change of said biological parameter further based, at least in part, on a rate of change of said biological parameter.

4. A system for assessing a likelihood of a patient to experience a cardiac arrhythmia, comprising:
a biological sensor configured to:
sense a biological parameter of said patient at a first time; then
sense said biological parameter of said patient at a second time, said second time being later than said first time;
a processor operatively coupled to said biological sensor and configured to determine a change of said biological parameter from said first time to said second time; and
wherein said system determines said likelihood of said patient experiencing a cardiac arrhythmia based, at least in part, on said change of said biological parameter.

5. The system of claim 4 wherein said processor determines said change of said biological parameter further based, at least in part, on a rate of change of said biological parameter.

6. The system of claim 4 wherein said first time and said second time occur after said patient has suffered a myocardial infarction.

7. The system of claim 4 wherein said first time and said second time occur after the patient has been hospitalized.

8. The system of claim 4 wherein said biological parameter is at least one of a T-wave alternan, a T-wave alternan associated with a baroreflex, heart rate turbulence, deceleration capacity and an ejection fraction of a heart of said patient.

9. The system of claim 8 wherein said likelihood of said patient experiencing a cardiac arrhythmia is relatively lower when a value of said T-wave alternan decreases from said first time to said second time than when said value of said T-wave alternan does not decrease from said first time to said second time.

10. The system of claim 8 wherein said likelihood of said patient experiencing a cardiac arrhythmia is relatively higher when a value of said T-wave alternan associated with said baroreflex decreases from said first time to said second time than when said value of said T-wave alternan associated with said baroreflex does not decrease from said first time to said second time.

11. The system of claim 8 wherein said likelihood of said patient experiencing a cardiac arrhythmia is relatively higher when a value of said heart rate turbulence changes less than a threshold value from said first time to said second time than when said value of said heart rate turbulence does not change less than said threshold value from said first time to said second time.

12. The system of claim 8 wherein said likelihood of said patient experiencing a cardiac arrhythmia is relatively higher when a value of said deceleration capacity decreases from said first time to said second time than when said value of said deceleration capacity does not decrease from said first time to said second time.

13. The system of claim 8 wherein said likelihood of said patient experiencing a cardiac arrhythmia is relatively higher when a value of said ejection fraction does not change greater than a threshold from said first time to said second time than when said value of said ejection fraction does not change greater than said threshold from said first time to said second time.

14. The system of claim 4 wherein said second time is approximately six hours after said first time.

15. The system of claim 4 wherein said system determines said change of said biological parameter based, at least in part, on a difference in a value of said biological parameter at said first time and said second time.

16. The system of claim 15 wherein said value of said biological parameter at said first time corresponds to a maximum value of said value of biological parameter measured over a first period of time and said value of said biological parameter at said second time corresponds to a maximum value of said biological parameter measured over a second period of time.

17. The system of claim 16 wherein each of said first period of time and said second period of time are approximately twenty-four hour periods.

18. The system of claim 15 wherein said value of said biological parameter at said first time corresponds to an average value of said biological parameter measured over a first period and said value of said biological parameter at said second time corresponds to an average value of said biological parameter measured over a second period of time.

19. The system of claim 18 wherein each of said first period of time and said second period of time are approximately two hour periods.

20. A device-implemented method for assessing a likelihood of a patient to experience a future cardiac arrhythmia with an implantable device system comprising an implantable sensor and a processor, comprising the steps of:
sensing a biological parameter of said patient with said sensor at a first time; then
sensing said biological parameter of said patient with said sensor at a second time; then
determining a change of said biological parameter from said first time to said second time with said implantable device system; and
determining said likelihood of said patient experiencing a cardiac arrhythmia with said implantable device system based, at least in part, on said change of said biological parameter.

21. The method of claim 20 wherein said determining said change step is based, at least in part, on a change of said biological parameter from said first time to said second time.

22. The method of claim 21 wherein said determining said change step is further based, at least in part, on a duration between said first time and said second time.

23. The method of claim 20 wherein each of said sensing steps occur after said patient has suffered a myocardial infarction.

24. The method of claim 20 wherein each of said sensing steps occur after said patient has been hospitalized.

25. The method of claim 20 wherein said biological parameter is at least one of a T-wave alternan, a T-wave alternan associated with a baroreflex, heart rate turbulence and deceleration capacity of a heart of said patient.

26. The method of claim 25 wherein said likelihood of said patient experiencing a cardiac arrhythmia is relatively lower when a value of said T-wave alternan decreases from said first time to said second time than when said value of said T-wave alternan does not decrease from said first time to said second time.

27. The method of claim 25 wherein said likelihood of said patient experiencing a cardiac arrhythmia is relatively higher when a value of said T-wave alternan associated with said baroreflex decreases from said first time to said second time than when said value of said T-wave alternan associated with said baroreflex does not decrease from said first time to said second time.

28. The method of claim 25 wherein said likelihood of said patient experiencing a cardiac arrhythmia is relatively higher when a value of said heart rate turbulence changes less than a threshold value from said first time to said second time than when said value of said heart rate turbulence does not change less than said threshold value from said first time to said second time.

29. The method of claim 25 wherein said likelihood of said patient experiencing a cardiac arrhythmia is relatively higher when a value of said deceleration capacity decreases from said first time to said second time than when said value of said deceleration capacity does not decrease from said first time to said second time.

30. The method of claim 25 wherein said likelihood of said patient experiencing a cardiac arrhythmia is relatively higher when a value of said ejection fraction does not change greater than a threshold from said first time to said second time than when said value of said ejection fraction does not change greater than said threshold from said first time to said second time.

31. The method of claim 20 wherein said second time is approximately six hours after said first time.

32. The method of claim 20 wherein said determining said change step is based, at least in part, on a difference in a value of said biological parameter at said first time and said second time.

33. The method of claim 32 wherein said value of said biological parameter at said first time corresponds to a maximum value of said value of biological parameter measured over a first period of time and said biological parameter at said second time corresponds to a maximum value of said biological parameter measured over a second period of time.

34. The method of claim 33 wherein each of said first period of time and said second period of time are approximately twenty-four hour periods.

35. The method of claim 32 wherein said value of said biological parameter at said first time corresponds to an average value of said biological parameter measured over a first period and said biological parameter at said second time corresponds to an average value of said biological parameter measured over a second period of time.

36. The method of claim 35 wherein each of said first period of time and said second period of time are approximately two hour periods.

37. The method as in claim 32 wherein said value of said biological parameter is measured continuously.
